SEQUENCE LISTING (64 sequences)

SEQ ID NO: 1 - DNA, 20 bp, Artificial Sequence
  Forward primer for amplifying a portion of Vascular endothelial growth factor-a (VEGFa)
agagcaacatcaccatgcag

SEQ ID NO: 2 - DNA, 20 bp, Artificial Sequence
  Reverse primer for amplifying a portion of vascular endothelial growth factor-a (VEGFa)
cagtgaacgctccaggattt

SEQ ID NO: 3 - DNA, 18 bp, Artificial Sequence
  Forward primer for amplifying  a portion of epidermal growth factor (EGF).
agctgtgtcttcttcact

SEQ ID NO: 4 - DNA, 19 bp, Artificial Sequence
  Reverse primer for amplifying a portion of epidermal growth factor (EGF).
tggggtcacctgctttaac

SEQ ID NO: 5 - DNA, 19 bp, Artificial Sequence
  Forward primer for amplifying a portion of keratinocyte growth factor (KGF).
cttccaatgaggtcagcaa

SEQ ID NO: 6 - DNA, 20 bp, Artificial Sequence
  Reverse primer for amplifying a portion of keratinocyte growth factor (KGF).
ccataaatcaacaggcaaaa

SEQ ID NO: 7 - DNA, 18 bp, Artificial Sequence
  Forward primer for amplifying a portion of insulin-like growth factor (IGF).
ggtggtttatgaatggtt

SEQ ID NO: 8 - DNA, 18 bp, Artificial Sequence
  Reverse primer for amplifying a portion of insulin-like growth factor (IGF).
agggtgtgtctaatggag

SEQ ID NO: 9 - DNA, 22 bp, Artificial Sequence
  Forward primer for amplifying a portion of heparin-binding EGF-like growth factor (HB-EGF).
aaaagaagaagaaaggaaaggg

SEQ ID NO: 10 - DNA, 19 bp, Artificial Sequence
  Reverse primer for amplifying a portion of heparin-binding EGF-like growth factor (HB-EGF).
tgcaagagggagtacggaa

SEQ ID NO: 11 - DNA, 19 bp, Artificial Sequence
  Forward primer for amplifying a portion of basic fibroblast growth factor (bFGF).
atgatgacgacgacgatga

SEQ ID NO: 12 - DNA, 21 bp, Artificial Sequence
  Reverse primer for amplifying a portion of basic fibroblast growth factor (bFGF).
ctacggtttggtttggtgttg

SEQ ID NO: 13 - DNA, 19 bp, Artificial Sequence
  Forward primer for amplifying a portion of transforming growth factor beta 1 (TGFb1).
tgttaaaactggcatctga

SEQ ID NO: 14 - DNA, 18 bp, Artificial Sequence
  Reverse primer for amplifying a portion of transforming growth factor beta 1 (TGFb1).
gtctcttaggaagtaggt

SEQ ID NO: 15 - DNA, 20 bp, Artificial Sequence
  Forward primer for amplifying a portion of stromal derived factor (SDF-1).
gtcctcttgctgtccagctc

SEQ ID NO: 16 - DNA, 20 bp, Artificial Sequence
  Reverse primer for amplifying a portion of stromal derived factor (SDF-1).
agatgcttgacgttggctct

SEQ ID NO: 17 - DNA, 18 bp, Artificial Sequence
  Forward primer for amplifying a portion of stem cell factor (SCF).
taatgttccccgctctct

SEQ ID NO: 18 - DNA, 22 bp, Artificial Sequence
  Reverse primer for amplifying a portion of stem cell factor (SCF).
ttttgctgtttttctttgcttt

SEQ ID NO: 19 - DNA, 18 bp, Artificial Sequence
  Forward primer for amplifying a portion of erythropoietin (EPO).
acagtcccagataccaaa

SEQ ID NO: 20 - DNA, 20 bp, Artificial Sequence
  Reverse primer for amplifying a portion of erythropoietin (EPO).
ggccttgccaaacttctatg

SEQ ID NO: 21 - DNA, 18 bp, Artificial Sequence
  Forward primer for amplifying a portion of granulocyte colony stimulating factor (G-CSF).
atcattctctccacttcc

SEQ ID NO: 22 - DNA, 22 bp, Artificial Sequence
  Reverse primer for amplifying a portion of granulocyte colony stimulating factor (G-CSF).
gtatttacccatctccttccct

SEQ ID NO: 23 - DNA, 21 bp, Artificial Sequence
  Forward primer for amplifying a portion of thrombopoietin-1 (TPO).
accccagactcctaaataaac

SEQ ID NO: 24 - DNA, 22 bp, Artificial Sequence
  Reverse primer for amplifying a portion of thrombopoietin-1 (TPO).
cagcagaacagggatagacaaa

SEQ ID NO: 25 - DNA, 19 bp, Artificial Sequence
  Forward primer for amplifying a portion of monocyte chemotactic protein-1 (MCP-1).
cccgtaaatctgaagctaa

SEQ ID NO: 26 - DNA, 22 bp, Artificial Sequence
  Reverse primer for amplifying a portion of monocyte chemotactic protein-1 (MCP-1).
cacactggtcactcctacagaa

SEQ ID NO: 27 - DNA, 18 bp, Artificial Sequence
  Forward primer for amplifying a portion of macrophage inflammatory protein 1a (MIP1a).
ccagtcccttttctgttc

SEQ ID NO: 28 - DNA, 19 bp, Artificial Sequence
  Reverse primer for amplifying a portion of macrophage inflammatory protein 1a (MIP1a).
cttggttgcagagtgtcat

SEQ ID NO: 29 - DNA, 18 bp, Artificial Sequence
  Forward primer for amplifying a portion of macrophage inflammatory protein 1b (MIP1b).
acgggggtcaattctaag

SEQ ID NO: 30 - DNA, 18 bp, Artificial Sequence
  Reverse primer for amplifying a portion of macrophage inflammatory protein 1b.
gccattcctgactccaca

SEQ ID NO: 31 - DNA, 22 bp, Artificial Sequence
  Forward primer for amplifying a portion of monokine induced by gamma interferon (MIG).
accaaaagaaaaagcaaaagag

SEQ ID NO: 32 - DNA, 18 bp, Artificial Sequence
  Reverse primer for amplifying a portion of monokine induced by gamma interferon (MIG).
ccttgaacgacgacgact

SEQ ID NO: 33 - DNA, 18 bp, Artificial Sequence
  Forward primer for amplifying a portion of procollagen, type I, alpha 1 (Col1a1).
attcggactagacattgg

SEQ ID NO: 34 - DNA, 18 bp, Artificial Sequence
  Reverse primer for amplifying a portion of procollagen, type I, alpha 1 (Col1a1).
gggttgttcgtctgtttc

SEQ ID NO: 35 - DNA, 18 bp, Artificial Sequence
  Forward primer for amplifying a portion of procollagen, type III, alpha 1 (Col3a1).
tttagacatgatgagctt

SEQ ID NO: 36 - DNA, 20 bp, Artificial Sequence
  Reverse primer for amplifying a portion of procollagen, type III, alpha 1 (Col3a1).
atctacgttggactgctgtg

SEQ ID NO: 37 - DNA, 20 bp, Artificial Sequence
  Forward primer for amplifying a portion of fibronectin-1 (Fn1).
aatccagtccacagccattc

SEQ ID NO: 38 - DNA, 20 bp, Artificial Sequence
  Reverse primer for amplifying a portion of fibronectin-1 (Fn1).
tagtggccaccatgagtcct

SEQ ID NO: 39 - DNA, 20 bp, Artificial Sequence
  Forward primer for amplifying a portion of laminin 1 (Lam1).
agtggaaggaatggttcacg

SEQ ID NO: 40 - DNA, 20 bp, Artificial Sequence
  Reverse primer for amplifying a portion of laminin 1 (Lam1).
tgccagtagccaggaagact

SEQ ID NO: 41 - DNA, 18 bp, Artificial Sequence
  Forward primer for amplifying a portion of tenascin C (TnC).
caagggagacaaggagag

SEQ ID NO: 42 - DNA, 20 bp, Artificial Sequence
  Reverse primer for amplifying a portion of tenascin C (TnC).
tcgtccagaaaaacgtcaga

SEQ ID NO: 43 - DNA, 18 bp, Artificial Sequence
  Forward primer for amplifying a portion of thrombospondin (Thbs1).
acaagtcacccagtccta

SEQ ID NO: 44 - DNA, 20 bp, Artificial Sequence
  Reverse primer for amplifying a portion of thrombospondin (Thbs1).
gagttcacaacctttacaga

SEQ ID NO: 45 - DNA, 20 bp, Artificial Sequence
  Forward primer for amplifying a portion of versican (PG-M).
tgtgcttcactcatcatttc

SEQ ID NO: 46 - DNA, 20 bp, Artificial Sequence
  Reverse primer for amplifying a portion of versican (PG-M).
gcagtcccataatccaaacc

SEQ ID NO: 47 - DNA, 18 bp, Artificial Sequence
  Forward primer for amplifying a portion of aggrecan 1 (Agc 1).
gagtgagaacctacggaa

SEQ ID NO: 48 - DNA, 21 bp, Artificial Sequence
  Reverse primer for amplifying a portion of aggrecan 1 (Agc1).
ctggggatgtcgcataaaaga

SEQ ID NO: 49 - DNA, 21 bp, Artificial Sequence
  Forward primer for amplifying a portion of decorin (PG II).
ctggcacagcataagtatatc

SEQ ID NO: 50 - DNA, 19 bp, Artificial Sequence
  Reverse primer for amplifying a portion of decorin (PG II).
agccgagtaggaagccttt

SEQ ID NO: 51 - DNA, 19 bp, Artificial Sequence
  Forward primer for amplifying a portion of hyaluronan synthase 1(Has1).
gggaggggtaatttattga

SEQ ID NO: 52 - DNA, 22 bp, Artificial Sequence
  Reverse primer for amplifying a portion of hyaluronan sythase 1 (Has1).
tagcaacagggagaaaatggag

SEQ ID NO: 53 - DNA, 19 bp, Artificial Sequence
  Forward primer for amplifying a portion of hyaluronan synthase2 (Has2).
caaaaagagcaccaaggtt

SEQ ID NO: 54 - DNA, 20 bp, Artificial Sequence
  Reverse primer for amplifying a portion of hyaluronan synthase 2(Has2).
gtgcagctttcccttagaca

SEQ ID NO: 55 - DNA, 20 bp, Artificial Sequence
  Forward primer for amplifying a portion of hyaluronan synthase 3(Has3).
gtttcttcccattcttcctc

SEQ ID NO: 56 - DNA, 18 bp, Artificial Sequence
  Reverse primer for amplifying a portion of hyaluronan synthase 3(Has3).
ccttgataatgcccacca

SEQ ID NO: 57 - DNA, 22 bp, Artificial Sequence
  Forward primer for amplifying a portion of angiopoietin-2 (Ang-2).
gacttccagaggacgtggaaag

SEQ ID NO: 58 - DNA, 21 bp, Artificial Sequence
  Reverse primer for amplifying a portion of angiopoietin-2 (Ang-2).
ctcattgcccagccagtactc

SEQ ID NO: 59 - DNA, 21 bp, Artificial Sequence
  Forward primer for amplifying a portion of angiopoietin 1 (Angpt1).
ttgtgattctggtgattgtgg

SEQ ID NO: 60 - DNA, 21 bp, Artificial Sequence
  Reverse primer for amplifying a portion of angiopoietin 1 (Angpt1).
cttgtttcgctttatttttgt

SEQ ID NO: 61 - DNA, 19 bp, Artificial Sequence
  Forward primer for amplifying a portion of gammainterferon-inducible protein-10 (CXCL10).
tgtcctagctctgtactgt

SEQ ID NO: 62 - DNA, 20 bp, Artificial Sequence
  Reverse primer for a portion of gamma interferon-inducible protein-10 (CXCL 10).
aacttagaactgacgagcct

SEQ ID NO: 63 - DNA, 21 bp, Artificial Sequence
  Forward primer for amplifying a portion of Actin beta.
acggccaggtcatcactattg

SEQ ID NO: 64 - DNA, 22 bp, Artificial Sequence
  Reverse primer for amplifying a portion of Actin beta.
caagaaggaaggctggaaaaga

(19) United States
(12) Patent Application Publication
(10) Pub. No.: US 2009/0136459 A1
(43) Pub. Date: May 28, 2009

(54) COMPOSITIONS FOR PREVENTING OR TREATING SKIN DEFECTS AND METHODS OF USE THEREOF

(76) Inventors: Yaojiong WU, Hong Kong (CN); Liwen CHEN, Toronto (CA); Edward E. Tredget, Edmonton (CA)

Correspondence Address: GARDNER GROFF GREENWALD & VILLANUEVA. PC, 2018 POWERS FERRY ROAD, SUITE 800, ATLANTA, GA, 30339, US

(21) Appl. No.: 12/107,439

(22) Filed: Apr. 22, 2008

Related U.S. Application Data
(60) Provisional application No. 60/913,576, filed on Apr. 24, 2007.

(51) Int. Cl. A61K 35/12; C12N 5/02

(52) U.S. Cl. 424/93.7; 435/391

(57) ABSTRACT

Described herein are compositions and methods that treat or prevent skin defects in a subject.

### CROSS REFERENCE TO RELATED APPLICATIONS

This application claims priority upon U.S. provisional application Ser. No. 60/913,576, filed Apr. 24, 2007. This application is hereby incorporated by reference in its entirety for all of its teachings.

### BACKGROUND

Optimum healing of a cutaneous wound requires a well-orchestrated integration of complex biological and molecular events of cell migration and proliferation, and extracellular matrix (ECM) deposition, angiogenesis, and remodeling. However, this orderly progression of the healing process is impaired in many chronic diseases such as, for example, diabetes. Common chronic skin wounds include diabetic foot ulcers, decubitus ulcers, and venous stasis ulcers, with diabetic ulcers being the most common cause of foot and leg amputation. Of the 150 million people with diabetes worldwide, 15% suffer from foot ulcerations, which often become non-healing chronic wounds.

Over the past decades little improvement has been shown in preventing morbidity and disability from chronic wounds. The best available treatment for these chronic wounds achieves only a 50% healing rate that is often temporary. Among the many factors contributing to non-healing wounds, decreased release of cytokines from inflammatory cells and fibroblasts and reduced angiogenesis are crucial. Recently, platelet-derived growth factor-BB (PDGF-BB) has been used in clinical trials in diabetic ulcers with the best result being a 15% increased incidence in wound closure at 20 weeks compared to conventional treatment. Clearly, there is a need for new therapies to improve healing of chronic wounds.

### SUMMARY

Described herein are compositions and methods that treat or prevent skin defects in a subject. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows mesenchymal stem cells (MSCs) promoted closure of excisional wounds in non-diabetic (A&B) or diabetic mice (C&D), increased re-epithelialization, structural regeneration and cellularity (E-G) compared to control medium or fibroblasts.

FIG. 2 shows MSCs engrafted into wounds differentiated into cytokeratin-expressing keratinocytes (A) and formed sweat/sebaceous gland-like structures (B&C).

FIG. 3 shows MSC-conditioned medium enhanced dermal keratinocyte growth (A), migration (B) and adhesion (C&D) compared to pre-conditioned medium or fibroblast-conditioned medium.

FIG. 4 shows MSC-treated wounds exhibited increased vascularity compared to vehicle medium- or fibroblast-treated wounds: (A) showing vasculature in wounds after whole skin mounts; (B) Immunostaining of wound sections showing endothelial cells; (C) Quantification of capillary density wounds.

FIG. 5 shows MSC-conditioned medium promoted HUVEC migration, proliferation (B) and tube formation compared to pre-conditioned medium or fibroblast-conditioned medium.

FIG. 6 shows the paracrine effect of MSCs in wound healing. (A) Real-Time PCR analysis shows expressional levels of cytokines and ECM molecules in MSCs vs. fibroblasts. (B) ELISA detection of IGF-1 in vehicle control, fibroblast- or BM-MSC-conditioned medium after hypoxic treatment. (C) RT-PCR analysis of IGF-1 in day 7 wounds. (D) Injection of MSC-conditioned medium promoted wound closure.

FIG. 7 shows an antibody array analysis of MSC- or fibroblast-conditioned medium showed distinctively different protein expression of cytokines.

FIG. 8 shows Western blot analysis indicating levels of VEGF-α, angiopoietin (Ang) 1 and 2 in fibroblast- or MSC-conditioned medium or treated wounds.

FIG. 9 is a FACS analysis indicating that MSC-treated wounds exhibited increased fractions of Flk-1+ or CD34+ cells and decreased fractions of CD3+ cells.

FIG. 10 shows immunostaining of wound sections showed that MSC-treated wounds had increased CD68+ macrophages and decreased CD3+ T cells compared to vehicle medium- or fibroblast-treated wounds.

FIG. 11 is (A) the FACS analysis of wound digests, which shows the fractions of DiI-keratinocytes in wounds receiving mixed DiI-keratinocytes and dermal fibroblasts (pink peak) or DiI-keratinocytes and BM-MSC (red peak) at 2 weeks, with a wound digest receiving no cell transplant was used as a negative control (grey peak); and (B) the average DiI-keratinocytes in wounds receiving mixed DiI-keratinocytes and dermal fibroblasts (pink peak) or DiI-keratinocytes and BM-MSC at 1 and 2 wk (n=4, P<0.01).

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

By “subject” is meant an individual. The subject can be a mammal such as a primate or a human. The term “subject” can include domesticated animals including, but not limited to, cats, dogs, etc., livestock (e.g., cattle, horses, pigs, sheep, goats, etc.), and laboratory animals (e.g., mouse, rabbit, rat, guinea pig, etc.).

By “contacting” is meant an instance of exposure by close physical contact of at least one substance to another substance. For example, contacting can include contacting a substance, such as a pharmacologic agent, with a cell.

“Treatment” or “treating” means to administer a composition to a subject or a system with an undesired condition (e.g., skin defect) to reduce the symptoms of the undesired condition. “Preventing” or “prevention” means eliminating the possibility of contracting the undesired condition (e.g., a skin defect). “Preventing” or “prevention” also includes decreasing the possibility of contracting the undesired condition.

By “effective amount” is meant a therapeutic amount needed to achieve the desired result or results.

“Induce” is defined as the initiation of a desired result or outcome. “Enhance” is defined as increasing or improving a pre-existing condition.

### I. Mesenchymal Stem Cells and Conditioned Medium

Described herein are mesenchymal stem cells, conditioned medium derived from mesenchymal stem cells or a combination thereof compounds that prevent or treat a skin defect present on a subject. Mesenchymal stem cells (MSC) are also referred to as bone marrow stromal cells or mesenchymal progenitor cells. MSCs useful herein are adherent to plastic under standard culture conditions, express CD105 and CD90 and lack expression of CD45, and can differentiate into osteoblasts, adipocytes and chondrocytes in vitro.

MSCs can be derived from a number of sources. For example, MSCs can be derived from non-bone marrow tissues such as, for example, fat and other tissues. In one aspect, the mesenchymal stem cells are derived from bone marrow. In one aspect, bone marrow (BM) MSCs are BM cells adherent to non-coated (or coated with extracellular matrix molecules) polystyrene or glass tissue culture dishes after culture for certain period of time (hours, days, weeks) in a medium supplemented with serum or sera derived from animals or humans with or without additional supplementation of growth factors or other nutrients. Bone marrow cells include all cells derived from bone marrow or a fraction of cells such as nucleated cells. The source of the bone marrow cells can also vary depending upon the subject. Thus, the bone marrow cells can be derived from humans, pigs, dogs, cats, mice, horses, and other mammals. In one aspect, the bone marrow derived mesenchymal stem cells can comprise endothelial progenitor cells.

The isolation and culture of mesenchymal stem cells are known in the art (Mackay et al., Tissue Eng. 4:415 428 (1988); William et al., Am Surg. 65:22 26 (1999); Pittenger et al., Science 284, 143-147 (1999)). Cells from bone marrow may be obtained by flushing the bone marrow or by washing ground bone pieces with a buffer or cell culture medium. BM-MSCs can also be isolated from a suspension of bone marrow cells through removal of blood lineage cells using immunodepletion. The isolated MSCs can then be expanded by plating the cells to new culture plates.

The phrase “conditioned medium derived from mesenchymal stem cells” is defined herein as a medium containing one or more components that were not present in the starting cell culture medium but produced by the culturing of the mesenchymal stem cells, where the new component or components enter the culture medium. Techniques for producing conditioned medium are known in the art. In general, the mesenchymal stem cells are placed on a support such as, for example, culture dishes, that the cells can adhere to. The cells are then incubated in a media that adequately feeds the cells for a sufficient time to grow the cells (e.g., from minutes up to weeks). The media can include one or more components for stimulating cell growth such as added chemicals, drugs, cytokine, and the like. The media can include amino-acids (both D and/or L-amino acids), sugars, deoxyribose, ribose, nucleosides, water soluble vitamins, riboflavin, salts, trace metals, lipids, acetate salts, phosphate salts, HEPES, phenol red, pyruvate salts, buffers, fat soluble vitamins (including A, D, E and K), steroids and their derivatives, cholesterol, fatty acids and lipids Tween 80, 2-mercaptoethanol pyramidines as well as a variety of supplements including serum (fetal, horse, calf, etc.), proteins (e.g., insulin, transferrin, growth factors, hormones, etc.) antibiotics, whole egg ultra filtrate, and attachment factors. In one aspect, the medium is serum-free. In another aspect, the media is DMEM, IMEM, or MEM.

By varying culture conditions, it is possible to vary the types of extracellular proteins (e.g., growth factors, cytokines, and stress proteins) that are secreted into the cell media as well as the relative amounts of each protein. In one aspect, the conditioned medium is produced under normoxic conditions during incubation. In another aspect, the conditioned medium is produced under hypoxic conditions during incubation, where minimal (less than 1%) to no oxygen is present during culturing. In this aspect, cell culturing of mesenchymal stem cells is performed in a chamber under anaerobic conditions. For example, an inert gas such as nitrogen can be used. In the case of bone marrow mesenchymal stem cells, high levels of several chemokines such as SDF-1 and stem cell factor (SCF) and cytokines such as EGF, IGF, KGF and VEGF can be expressed by mesenchymal stem cells under hypoxic conditions. These factors have been known important for the migration, adhesion and proliferation of keratinocytes and endothelial cells. In one aspect, bone marrow mesenchymal stem cells produce higher expression levels of IGF-1, EGF and KGF and lower expression of TGF-β1 compared to fibroblasts, which have been known to mediate scar repair.

It is contemplated that the conditioned medium can be further processed once it is prepared and isolated. For example, the conditioned medium can be concentrated by a water flux filtration device or by ultrafiltration. In other aspects, the conditioned medium can be further purified to remove undesirable impurities. Methods of purification include, but are not limited to, gel chromatography (using matrices such as sephadex) ion exchange, metal chelate affinity chromatography with an insoluble matrix such as cross-linked agarose, HPLC purification and hydrophobic interaction chromatography of the conditioned media.

### II. Pharmaceutical Compositions

In one aspect, any of the mesenchymal stem cells and conditioned medium described above can be formulated into a pharmaceutical composition. The pharmaceutical compositions can be prepared using techniques known in the art. In one aspect, the composition is prepared by admixing the cells or conditioned medium described herein with a pharmaceutically-acceptable carrier.

It will be appreciated that the actual preferred amounts, modes of administration, and administration intervals of the mesenchymal stem cells and conditioned medium in a specified case will vary according to the specific composition being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity-enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

The pharmaceutical composition can be administered in a number of ways depending on whether local or systemic treatment is desired, and on the area to be treated. Administration can be topically (e.g., dermal, ophthalmical, vaginal, rectal, intranasal). Formulations for topical administration can include ointments, lotions, creams, gels, drops, suppositories, sprays, liquids and powders. Conventional pharmaceutical carriers, aqueous, powder or oily bases, thickeners and the like can be necessary or desirable. Formulations for topical administration can also include the use of other carriers such as collagens (including gelatin), elastins, laminins, fibronectin, hyaluronic acid, proteoglycans and glycosaminoglycans.

Preparations for parenteral administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

The pharmaceutical compositions can also include other drugs and biologically-active agents. The biologically-active agent is capable of providing a local or systemic biological, physiological or therapeutic effect in the biological system. For example, the agent can act to control infection or inflammation, enhance cell growth and tissue regeneration, control tumor growth, act as an analgesic, promote anti-cell attachment, and enhance bone growth, among other functions.

### III. Methods of Use

Described herein are methods for preventing or treating skin defects in subject using mesenchymal stem cells, a conditioned medium derived from mesenchymal stem cells, or a combination thereof. The method comprises administering to the skin defect a composition comprising mesenchymal stem cells, a conditioned medium derived from mesenchymal stem cells, or a combination thereof.

A number of different skin defects can be treated or prevented using the techniques described herein. A “skin defect” as defined as any undesirable condition to any part of the skin, which includes the epidermis, the dermis, and all structures associated or present in the epidermis and dermis. In one aspect, the methods described herein can be used in cosmetic applications. For example, the methods can reduce or prevent wrinkles, scar formation (e.g., normal scars and hypertrophic scars), aging spots, or frown lines. In one aspect, the mesenchymal stem cells, a conditioned medium derived from mesenchymal stem cells, or a combination thereof can be a topical formulation that can be applied to directly to the skin. Thus, the composition and methods described herein can be used as anti-aging agents. Not wishing to be bound by theory, it is believed that the MSCs promote cutaneous regeneration through differentiation and paracrine mechanisms.

In other aspects, the skin defect can be a serious skin wound such as an incision or ulcer. Diabetic ulcers and other chronic wounds are difficult to heal and little improvement has been shown in preventing morbidity and disability in the past few decades. The best available treatment for chronic wounds achieves only a 50% healing rate that is often temporary. Moreover, injury to the skin and other tissues heals not by the regeneration of the tissue to the pre-injured form but by the formation of scar tissue.

In situations where the skin defect is an incision, ulcer, or seriously damaged dermal tissue, the mode of administration of the mesenchymal stem cells and/or conditioned medium can vary. In one aspect, the mesenchymal stem cells and/or conditioned medium can be administered directly to the wound via injection or by topical application. In other aspects, the mesenchymal stem cells and/or conditioned medium can be applied to a bandage that comes into contact with the wound. Alternatively, the mesenchymal stem cells and/or conditioned medium can be incorporated into a hydrogel or other forms of matrix materials, such as a sheet made of collagen (including gelatin), elastin, laminin, fibronectin, hyaluronic acid, proteoglycans, glycosaminoglycans, or any combination thereof, which can then be topically applied to or inserted into the wound. It is contemplated that additional cell-types can be added to the mesenchymal stem cells and/or conditioned medium prior to administration to the subject. In one aspect, keratinocytes, fibroblasts, or endothelial cells can be added to the mesenchymal stem cells and/or conditioned medium in order to facilitate and expedite wound healing. The methods described above involve the in vivo administration of mesenchymal stem cells and/or conditioned medium to treat or prevent a skin defect. In vitro and ex vivo applications are also contemplated using the methods described herein and demonstrated in the Examples.

The methods described herein provide additional advantages with respect to wound healing. Wounds treated with bone marrow mesenchymal stem cells or cultured medium accelerate wound closure. For example, as will be shown in the Examples, bone marrow mesenchymal stem cells can differentiate into keratinocytes in the wound. In addition, bone marrow mesenchymal stem cells conditioned medium can promote keratinocyte migration, growth and adhesion and endothelial cell tube formation. Thus, the methods described herein result in the formation of dermal epithelial cells and avoid scar formation. The use of bone marrow mesenchymal stem cells or cultured medium derived therefrom can also result in the formation of sweat and sebaceous gland-like structures as well as hair follicles.

Additionally, the methods described herein can induce or enhance angiogenesis in the wound. Neovascularization is an important step in the wound healing process. The formation of new blood vessels is necessary to sustain the newly formed granulation tissue and the survival of keratinocytes. Angiogenesis is a complex process that relies on ECM in the wound bed as well as migration and mitogenic stimulation of endothelial cells. In one aspect, bone marrow mesenchymal stem cells cultured medium contains high levels of several known pro-angiogenic factors such as VEGF, bFGF and IGF and Ang1. In addition, the conditioned medium contains higher amounts of chemoattractive factors such as, for example, MIP and MIG, for attracting macrophages to the wound. In certain aspects, wounds treated with bone marrow mesenchymal stem cells or cultured medium derived therefrom have higher amounts of endothelial progenitor cells, which are cells associated with angiogenesis. Both endothelial progenitor cells and macrophages play crucially roles in wound healing. Reduced presence of endothelial progenitor cells are associated with impaired wound healing. In the absence of macrophages, wounds do not close.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Methods

All animal procedures were approved under the guidelines of the Health Sciences Animal Policy and Welfare Committee of the University of Alberta.

### Isolation and Purification of MSCs

Bone marrow was collected from the femurs of 5-7 week-old male C57 or C57 GFP transgenic (C57BL/6 TgN[ACT6EGFP]) mice (Jackson Laboratory). The mononuclear fraction of the bone marrow was isolated with a Ficoll-paque density gradient. The nucleated cells were plated in plastic tissue culture dishes and incubated in minimal essential medium (α-MEM; GIBCO) supplemented with 17% fetal bovine serum (FBS). BM-MSCs were first selected by their adherent property preferentially attaching to uncoated polystyrene tissue culture dishes and further purified by immunodepletion using magnetic micro beads (Miltenyi Biotec) and monoclonal antibodies against CD34, CD14, Gr1, CD3 and CD19. Passage 3-5 cells were used for the experiments.

### Flow Cytometry

Passage 3 BM-MSCs were detached with trypsin/EDTA, neutralized with MSC growth medium, washed with phosphate-buffered saline (PBS) and resuspended in PBS containing 1% bovine serum albumin (BSA) at 106/mL. 100 μL cell aliquots were incubated with fluorescein isothiocyanate (FITC)- or phycoerythrin (PE)-conjugated monoclonal antibodies specific for Sca-1, CD 105 (endoglin), CD29, CD44, CD90, CD45, CD14, CD3, CD19 and CD34, or control isotype IgG on ice for 30 minutes. Cells were washed with PBS. All antibodies were purchased from BD Pharmingen. For detection of GFP⁺ cells in the skin, excised murine skin and wounds were dispersed into single cell suspension. In brief, the tissue was incubated with dispase I (Sigma) at 1 mg/ml overnight at 4° C., minced and incubated in a digestion buffer containing hyaluronidase 1 mg/ml, collagenase D 1 mg/ml and DNAase 150 units/ml in 37° C. shaking water bath for 2 hours. The dispase digest and the hyauluronidase digest were pooled and filtered through 70 um Nylon cell strainer. Cells were washed, pelleted and resuspended in PBS containing 3% FBS. 10 000 events were analyzed by flow cytometry (Becton Dickinson) using Cell Quest software.

### MSC Differentiation Assay

Passage 4 BM-MSCs were tested for their ability to differentiate into adipocytes, osteoblasts, and chondrocytes. For adipocyte differentiation, cells were cultured for 3 weeks in adipogenic medium containing 10-6 M dexamethasone, 10 μg/mL insulin, and 100 μg/mL 3-isobutyl-L-methylxantine (Sigma). For osteoblast differentiation, cells were cultured for 3 weeks in osteogenic medium containing 10-7 M dexamethasone, 50 μg/mL ascorbic acid, and 10 mM β-glycerophosphate (Sigma). The cultures were stained for alkaline phosphatase (alkaline phospatase detection kit, Sigma) or with Alizarin Red (Sigma). For chondrocyte differentiation, a pellet culture system was used. The pellet was cultured for 3 weeks in DMEM (high glucose) containing 10-7 M dexamethasone, 50 μg/mL ascorbate-2-phosphate, 100 μg/mL pyruvate (Sigma), 10 ng/mL TGF-β1 (R&D Systems) and 50 mg/mL ITS+Premix (BD Biosciences, 6.25 μg/mL insulin, 6.25 μg/mL transferrin, 6.25 ng/mL selenious acid, 1.25 mg/mL bovine serum albumin, and 5.35 mg/mL linoleic acid). The cultures were fixed and sectioned for alcian blue (Sigma) stain or subjected to RNA extraction and RT-PCR analysis.

Isolation of Cells from the Skin

Dermal keratinocytes were isolated from neonatal Balb/C mouse skin. In brief, the skin was incubated with Dispase II (Sigma) in keratinocyte-SFM (Gibco) at 10 mg/ml for 13 hours at 4° C. After separation from the dermis, the epidermis was trypsinized (0.25% trypsin/0.1% EDTA) for 10 minutes. Cells were seeded on plastic tissue culture plates in keratinocyte-SFM supplemented with 10 ng/ml EGF and 10⁻¹⁰ M choleratoxin. Fibroblasts were obtained from the dermis of neonatal Balb/C mice after digestion with 0.75% collagenase and cultured in DMEM supplemented with 10% FBS. Passage 3-5 cells were used for the experiments, Wound healing model and BM-MSC transplantation.

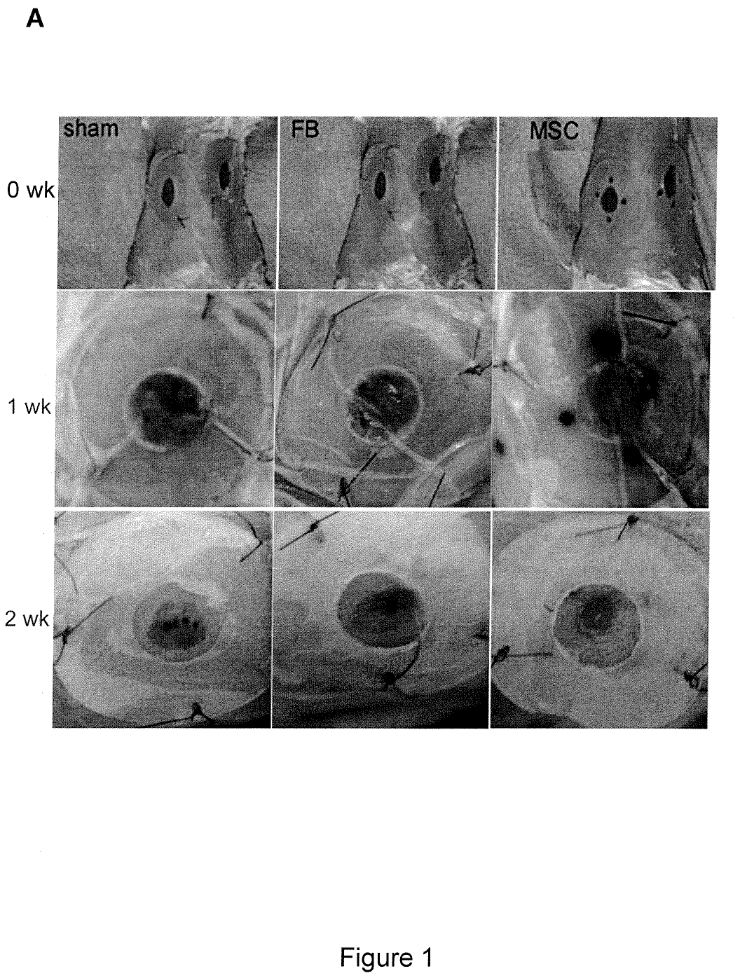

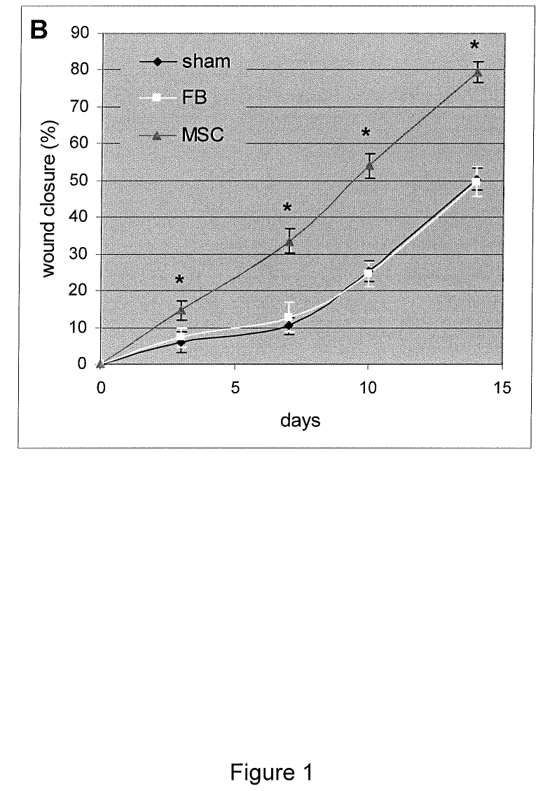

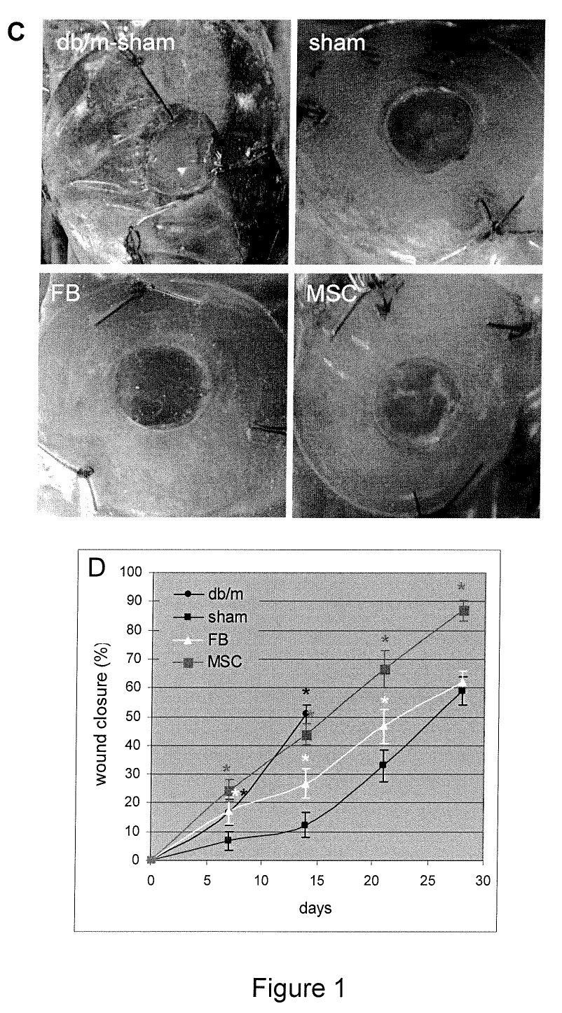

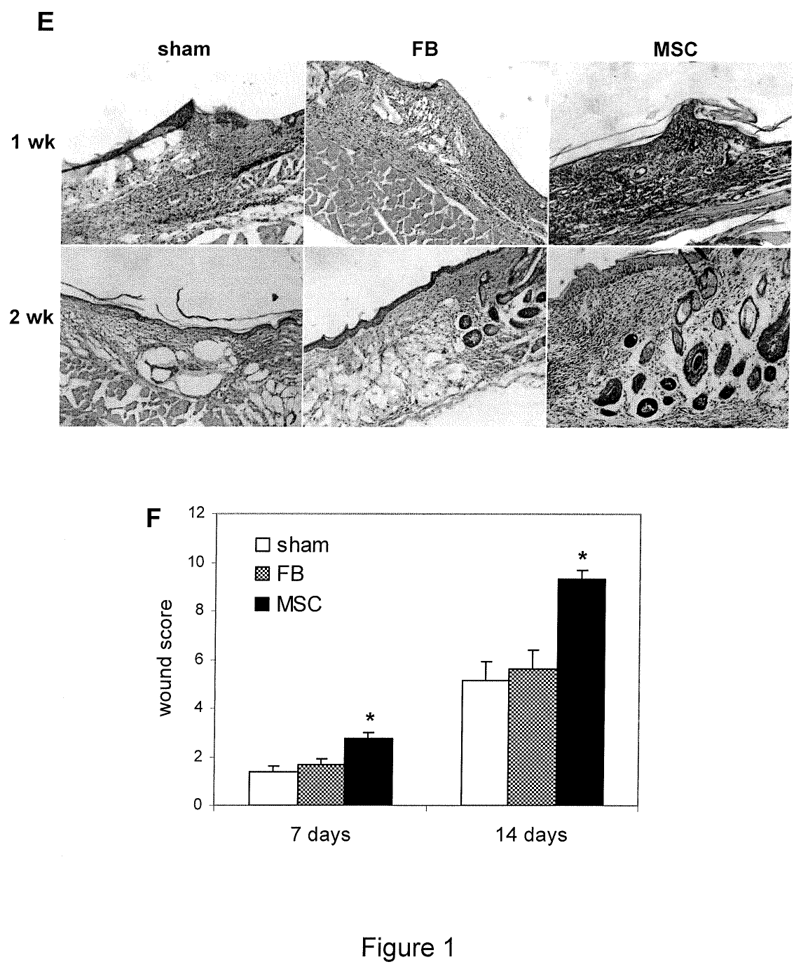

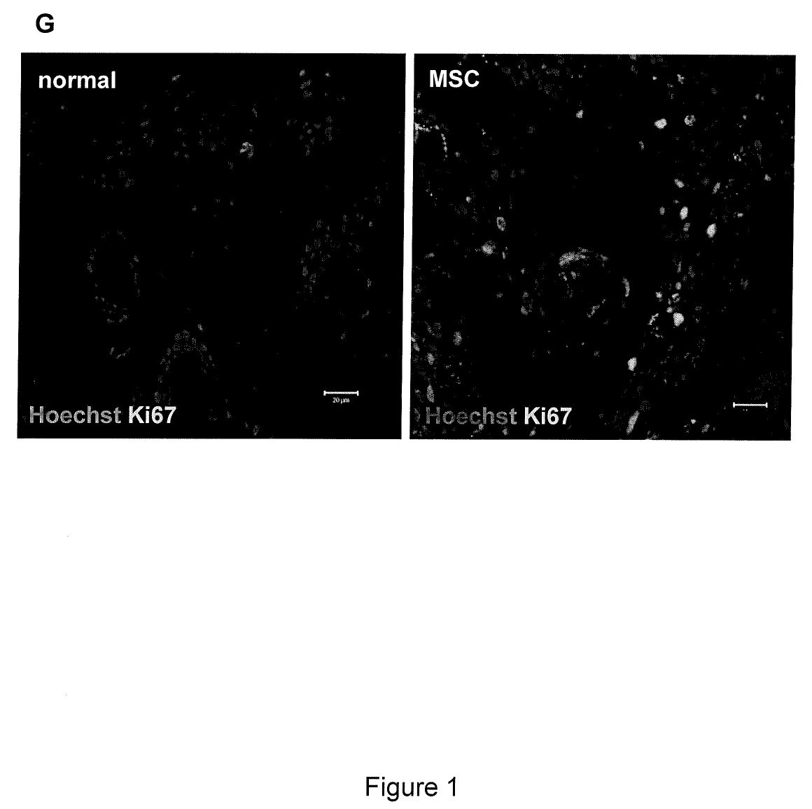

Balb/C mice (8 week-old, female, body weight 20-23 grams), db/db mice (BKS.Cg-m+/+Lepr^{db}/J, db⁺/db⁺, 13 week-old, female) and their normal littermates (db⁺/m⁺, 13 weeks old, female) were obtained from Jackson Laboratory (Table 1). At the initiation of the experiments, db/db mice exhibited significantly increased body weight, blood glucose, triglyceride and cholesterol compared to db⁺/m⁺ mice. The animals were randomly divided into three groups and the excisional wound-splinting model was generated. In brief, after hair removal from the dorsal surface and anesthesia, two 6-mm full-thickness excisional skin wounds were created on each side of the midline. Each wound received implantation of one million cells (allogeneic murine GFP⁺ BM-MSCs or allogeneic neonatal dermal fibroblasts): 0.7×10⁶ in 60 μl phosphate buffered saline (PBS) was injected intradermally around the wound at 4 injection sites and 0.3×10⁶ in 20 μl Growth Factor Reduced (GFR) Matrigel BD, which is composed of collagens, laminin, elastin, and proteoglycans, was applied onto the wound bed. A donut-shaped silicone splint was placed so that the wound was centered within the splint. An immediate-bonding adhesive (Krazy Glue®) was used to fix the splint to the skin followed by interrupted sutures to stabilize its position (FIG. 1A) and Tegaderm (3M) was placed over the wounds. The animals were housed individually. We tested the adhesive (Krazy Glue®) on the skin in mice prior to this experiment and did not observe any skin irritation or allergic reaction.

### Wound Analysis

Digital photographs of wounds were taken at day 0, 3, 7, 10, 14, 21 and 28. Time to wound closure was defined as the time at which the wound bed was completely reepithelialized and filled with new tissue. Wound area was measured by tracing the wound margin and calculated using an image analysis program (NIH Image). The individual measuring samples was blinded as to the group and treatment. The percentage of wound closure was calculated as: (area of original wound−area of actual wound)/area of original wound×100. The inside edge of the splint exactly matched the edge of the wound, so that the splinted hole was used to represent the original wound size. Mice were sacrificed at 7, 14 and 28 days when skin samples including the wound and 4 mm of the surrounding skin were harvested using a 10 mm punch biopsy. Each wound was bisected into two pieces, which were used for histology and RNA extraction. For whole skin mount, the entire wound and surrounding skin was placed on plastic (tissue culture dish) with the dermis side down and photographed immediately.

### Histologic Examination

All tissue specimens were fixed in 3% freshly prepared paraformaldehyde (PFA) for 24 h and embedded in OCT. Six-micron-thick sections were stained with hematoxylin and eosin (H&E) for light microscopy. Histological scoring was performed in a blinded fashion. Each slide was given a histological score ranging from 1 to 10 according to the following parameters modified from previous reports: reepithelialization, dermal cellularity and regeneration, granulation tissue formation and angiogenesis. Capillary density was assessed morphometrically by examining 3 fields per section (6 μm thick) of the wound between the edges in 6 successive sections after immunofluorescence staining for endothelial cells with an anti-CD31 antibody. The criteria used for histological scores of wound healing are reepithelialization, cellularity, granulation formation and angiogenesis (Table 2).

For immunofluorescence, tissue sections were pre-incubated with sodium borohydride (1 mg/ml in PBS) to reduce auto-fluorescence. Endogenous biotin was blocked with streptavidin biotin blocking kit (Vector). Keratinocytes were stained with an antibody against a variety of epidermal keratins (keratin subunits of 58, 56, 52, 60, 51 and 48 kD, Dako, Wide Spectrum Screening, WSS). GFP was detected with a goat anti-GFP antibody (USbiological) and visualized with FITC-conjugated secondary antibody against goat. Endothelial cells were identified with an antibody against CD31 or Von Willebrand factor (vWF, BD Biosciences) followed by incubation with a biotinyolated secondary antibody (Jackson Immunoresearch) and visualized with Fluor 568-conjugated streptavidin (Invitrogen). Nuclei staining with Hoeschst and Ki67 or isotype IgG (Dako) was performed. For negative controls in CD31 and Ki67 immunostaining, isotype control antibody for each was used. In negative controls for GFP and cytokeratin immunostaining, sections were treated with FITC- or TRITC-conjugated secondary antibody alone. Sections were examined with a Zeiss LSM 510 confocal microspore. The percentages of Ki67-positive nuclei was determined by examining 5 fields covering the epidermis and the underlying dermis per section of the wound between the edges in 4 successive sections and the total number of nuclei per field was counted using an image analysis program (NIH Image). Appendage-like structures in the wounds were photographed and the numbers of the structures per section with over 10% Ki67-postive nuclei were counted.

### Conditioned Medium

Conditioned medium was generated as follows: 80% confluent passage 3 BM-MSCs or neonatal dermal fibroblasts in 10 cm-tissue culture dishes were fed with 5 ml of serum-free α-MEM or other media as indicated per dish and incubated for 13 h under normoxic or hypoxic conditions (5% CO₂, 95% N₂, and 0.5% O₂) in a hypoxic chamber. For in vivo experiments, the conditioned medium was further concentrated by ultrafiltration using Centrifugal Filter Units with 5 kDₐ cut-off (Millipore) following manufacturer's instructions.

### Cell Growth and Adhesion

Equal numbers of murine dermal keratinocytes or human umbilical vein endothelial cells (HUVECs, CAMBREX) were seeded in 12-well tissue culture plates in vehicle-, FB- or MSC-conditioned medium or control medium as indicated and incubated for various times. Media were changed once at day 4. Cells were detached and counted. In the adhesion assay, 12-well tissue culture plates were coated with BSA, fibronectin (10 ng/ml), vehicle medium, or FB- or MSC-conditioned serum-free α-MEM. The plates were washed with PBS and blocked with 5% BSA for 1 h. After washing, 10⁵ cells/well were seeded in keratinocyte growth medium and incubated for 1 h at 37° C. After removal of unattached cells, attached cells were detached by trypsinization and counted.

### Cell Migration

Cell transwell migration assay was performed where 0.5×10⁵ keratinocytes or HUVECs per well in 100 μl medium were added to the top chambers of 24-well transwellplates (8.0 μm, pore size; Costar). 600 μl MSC- or fibroblast (FB)-conditioned medium or vehicle control medium was added to the lower chambers. Cells were maintained at 37° C. for 8 (HUVECs) or 15 (keratinocytes) hours. Cells on the upper side of the filter were wiped out and cells on the bottom side of the filter (migrated) were fixed with PFA, stained with Hoechst to visualize nuclei and photographed. The numbers of cells in 6 fields per well were counted.

### Endothelial Cell Network Formation Assay

2.5×10⁴ HUVECs per well were suspended in 0.4 ml EGM-2 (basal plus growth factor and FBS supplements, CAMBREX), vehicle, fibroblast- or BM-MSC-conditioned EGM-2 basal medium supplemented with 0.75% FBS, seeded onto Matrigel (BD)-coated 24-well plates and incubated at 37° C./5% CO₂ for 12 h. After removal of the media, the cells were fixed and images were captured. The total length of the tube-like structures was determined using NIH-Image software. Four random fields were measured for each well.

Co-Culture of BM-MSCs with Keratinocytes

Murine or human keratinocytes were cultured on 4-well chamber slides to 80% confluence and then irradiated with 10 Gy from a ⁶⁰Co source at a dose rate of 0.3 Gy/min. Alternatively, keratinocyte monolayers were fixed with 1% PFA for 0.5 h followed by extensive washes with PBS. 24 h later, 10⁴ or 2×10⁴ GFP⁺ BM-MSCs were seeded on the keratinocyte monolayer and maintained in K-SFM supplemented with 0, 1, 2.5 and 5% FBS for 1 or 2 weeks. Medium was changed every 3 days. Cells were fixed and stained with an antibody reacting to epidermal keratins (Dako, WSS).

### Real-Time PCR Analysis

Total RNA was extracted (RNeasy Mini Kit, Qiagen) from BM-MSCs or neonatal dermal fibroblasts, which were 80% confluent and treated in hypoxic conditions for 8 h. For tissue total RNA, fresh skin tissues were immediately preserved in RNAlater (Qiagen) followed by tissue homogenization and total RNA extraction using affinity resin columns (Qiagen). Total RNA was reverse transcribed using SuperScript First-Strand Synthesis kit (RT-PCR; Invitrogen). The primers used for Real-Time PCR are shown in Table 3. Reactions were performed using SYBR-Green PCR master mix (Applied Biosystems) in BioRad iCycler iQ Detection System. As an internal control, levels of beta-actin were quantified in parallel with target genes. Normalization and fold change were calculated using the ΔΔCt method.

### Statistical Analysis

All values are expressed as mean±SD. Student's paired t test was performed for comparison of data of paired samples and one-way ANOVA test was used for multiple group comparisons. A probability (P) value <0.05 was considered significant.

### Results

### BM-MSCs Enhance Wound Healing

Fluorescence activated cell sorting (FACS) analysis of the BM-MSCs indicated that they had typical features of MSCs. They were negative for lineage cell markers such as CD34, CD45, CD14, CD3 and CD19 and strongly expressed typical surface antigens for MSCs such as Sca-1, CD29, CD44, CD105, and CD90 (data not shown). When cultured in adipogenic, osteogenic or chondrogenic medium, they differentiated into adipocytes, osteoblasts, or chondrocytes (data not shown).

BM-MSC-treated wounds exhibited accelerated wound closure in Balb/C mice (FIGS. 1A&B) and genetically diabetic db/db mice (FIGS. 1C&D) compared to fibroblast- or vehicle medium-treated wounds. The enhancement appeared early at three days after implantation in Balb/C mice and became more evident after 7 days in both Balb/C and db/db mice. Wound closure at day 7 in BM-MSC-treated db/db mice appeared even faster than in vehicle medium-treated non-diabetic db/m mice (FIG. 1D), although this pattern did not last to day 14 when wound closure in vehicle medium-treated db/m mice surpassed BM-MSC-treated db/db mice. As splints in some db/m mice were not tightly adherent to the skin and failed to restrict skin contraction after 14 days due to movement and hair re-growth, data in wound closure after day 14 was excluded. In contrast, splints remained firmly adherent to the skin in db/db mice due to dramatically reduced physical movement and decreased hair re-growth. Fibroblast-treatment accelerated wound closure in db/db mice at day 7, 14 and 21 (P<0.01) but not at day 28 and in Balb/C mice compared with vehicle medium-treatment.

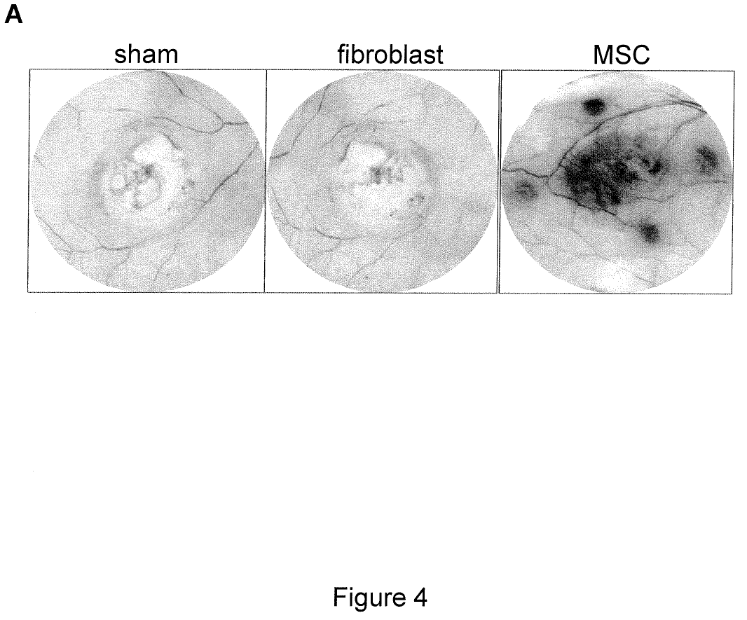

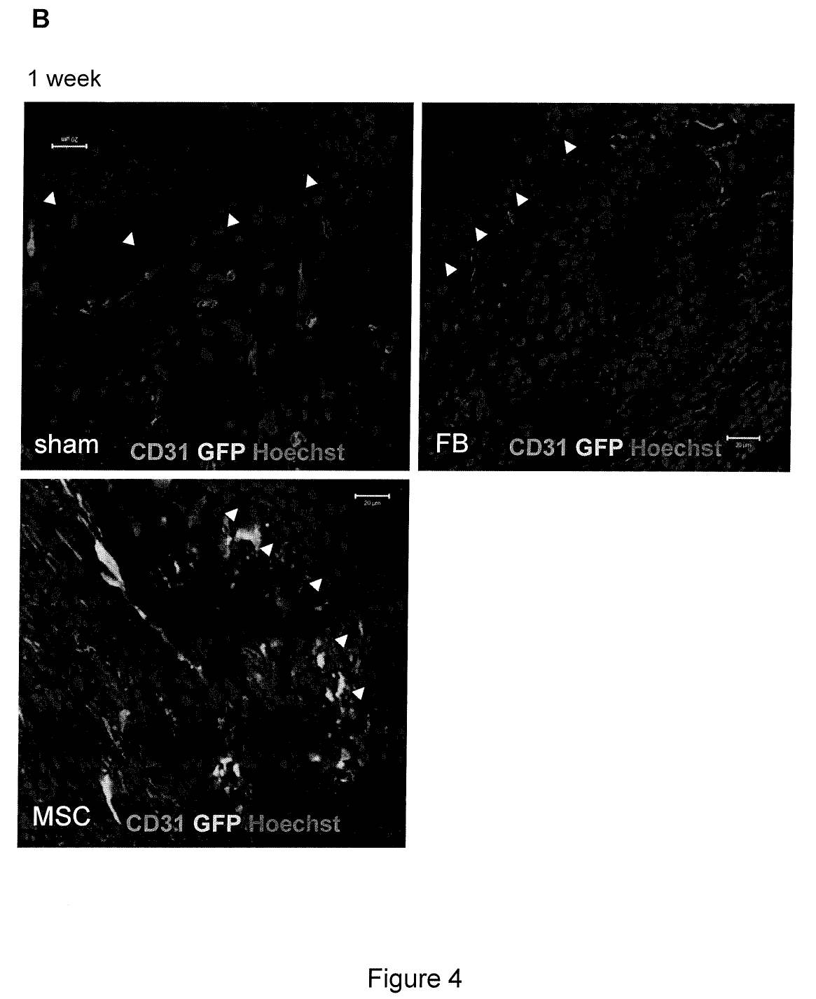

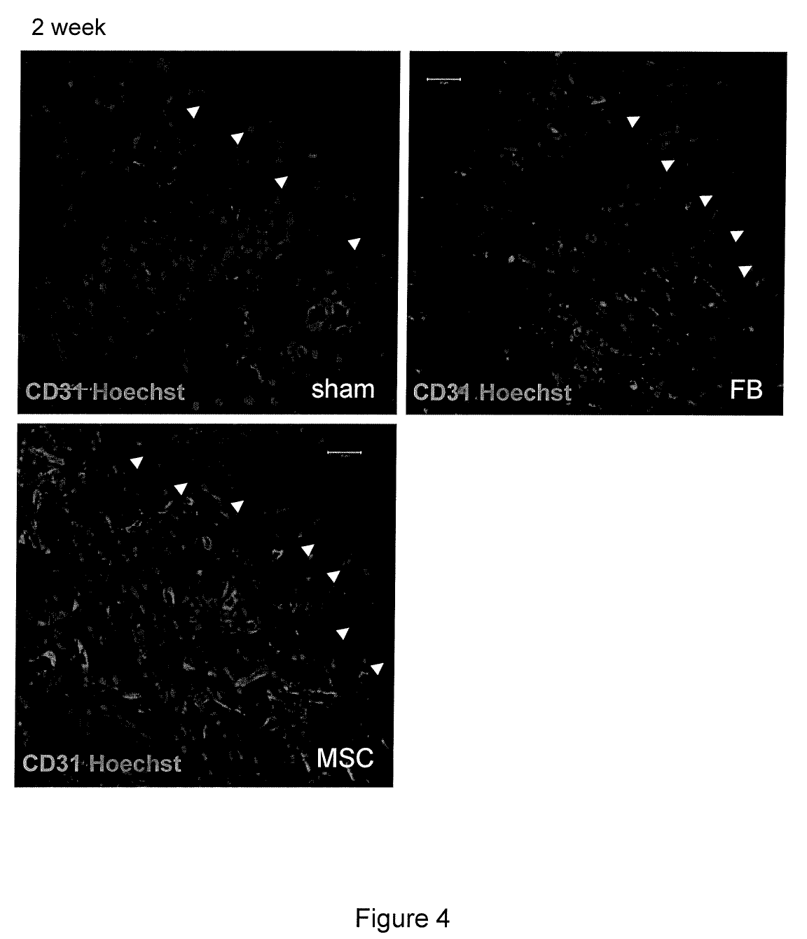

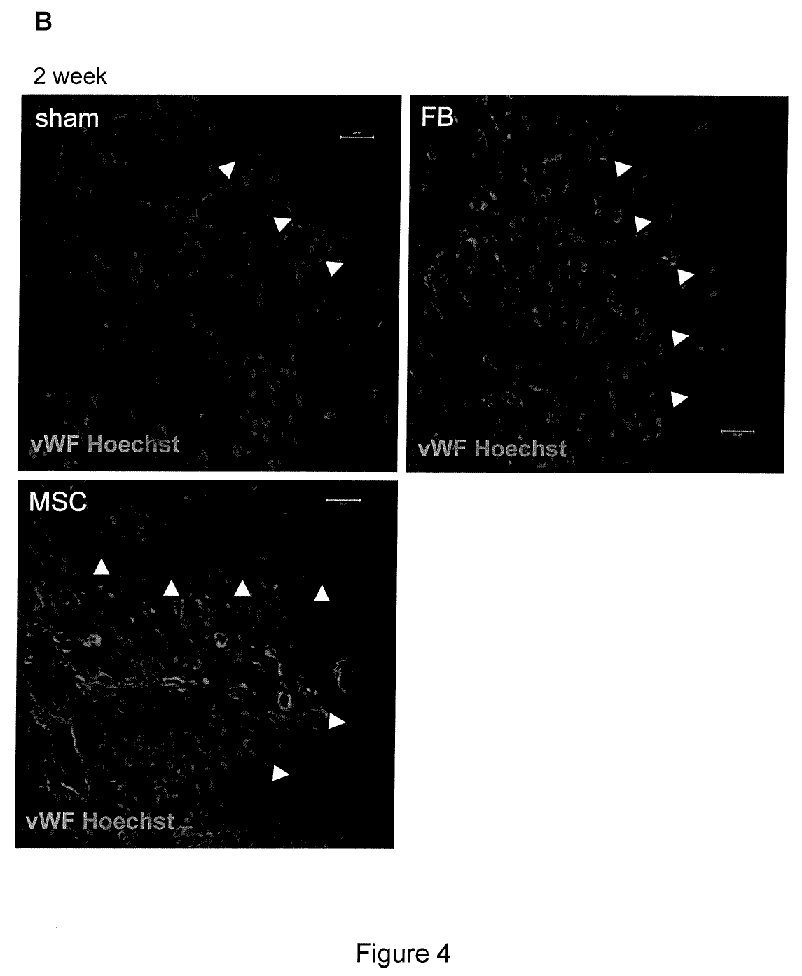

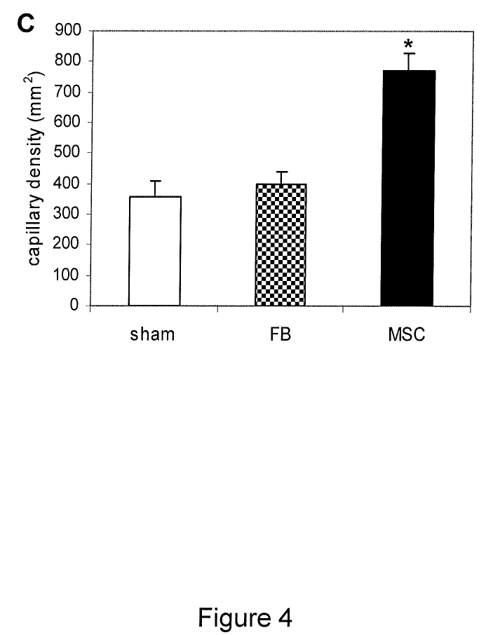

Histological evaluation of wounds in Balb/C mice at day 7 disclosed enhanced reepithelialization in BM-MSC-treated wounds (complete epithelialization in all 10 wounds examined, n=5) compared with fibroblast-treated wounds (complete reepithelialization in 6 of 10 wounds, n=5) or vehicle medium-treated wounds (complete reepithelialization in 4 of 10 wounds, n=5). Analysis of day 7 and 14 wounds indicated that BM-MSC-treated wounds had enhanced cellularity, thicker and larger granulation tissue, increased vasculature (also see FIGS. 4B&C) and greater numbers of developing hair follicle- or gland-like structures (FIGS. 1E&F). The developing hair follicle- or gland-like structures in the dermis of wounds exhibited increased proportions of Ki67-positive cells (FIG. 1G).

### BM-MSCs Contribute to Dermal Keratinocytes and Appendages

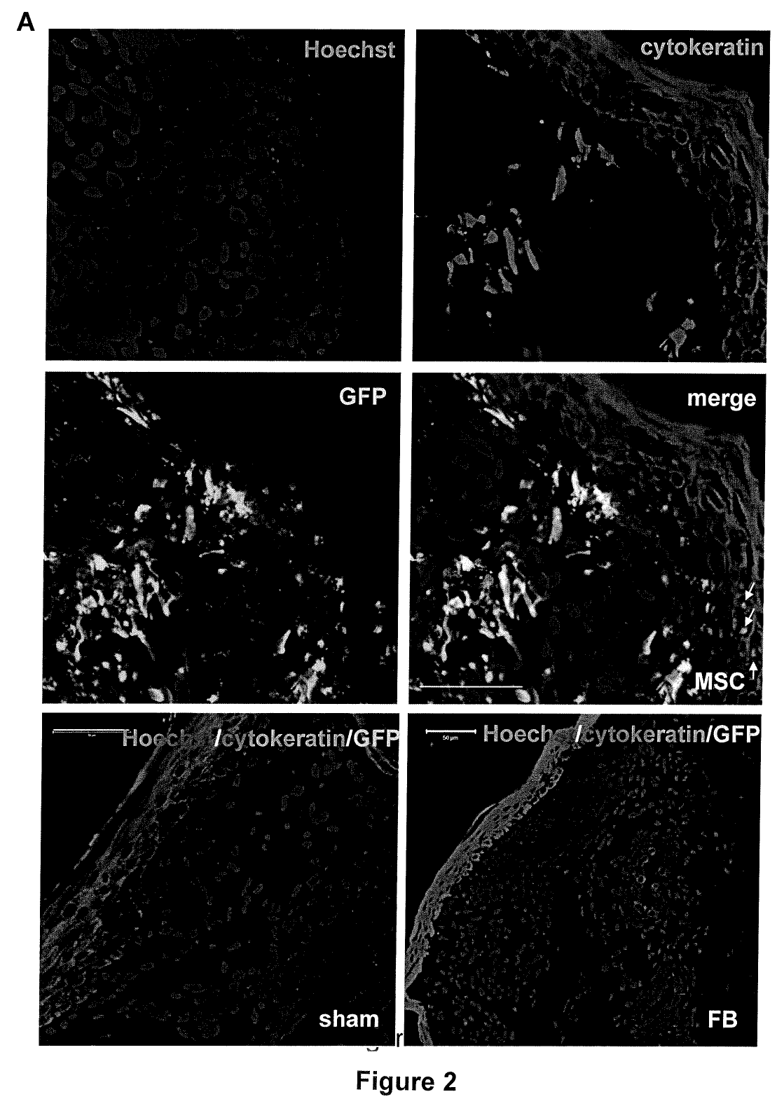

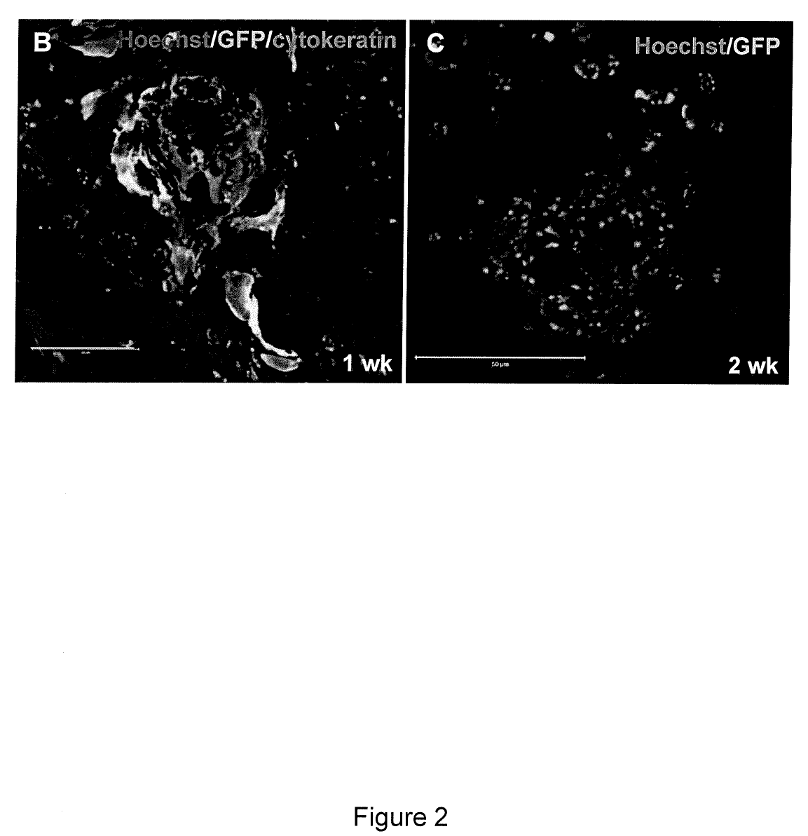

Immunostaining of wound sections showed that 7 days after surgery, in wounds receiving GFP⁺ BM-MSCs, there were large numbers of GFP-positive MSCs, of which many co-expressed keratinocyte-specific keratins (FIG. 2A). Most of the GFP and cytokeratin double positive cells were found in the dermis adjacent to the epidermis, but some appeared in the epidermis, particularly the basal stratum (FIG. 2A). GFP positive cells were not detected in vehicle medium- or fibroblast-treated wounds (FIG. 2A), indicating specificity of our staining. BM-MSCs in the dermis of the wounded skin formed gland-like structures, which were positive for cytokeratin (FIG. 2B). The structures appeared more like sweat glands at day 14 (FIG. 2C). The overall abundance of GFP-positive BM-MSCs in day 14 wounds decreased.

### BM-MSC-Conditioned Medium Promotes Keratinocyte Growth, Migration and Adhesion

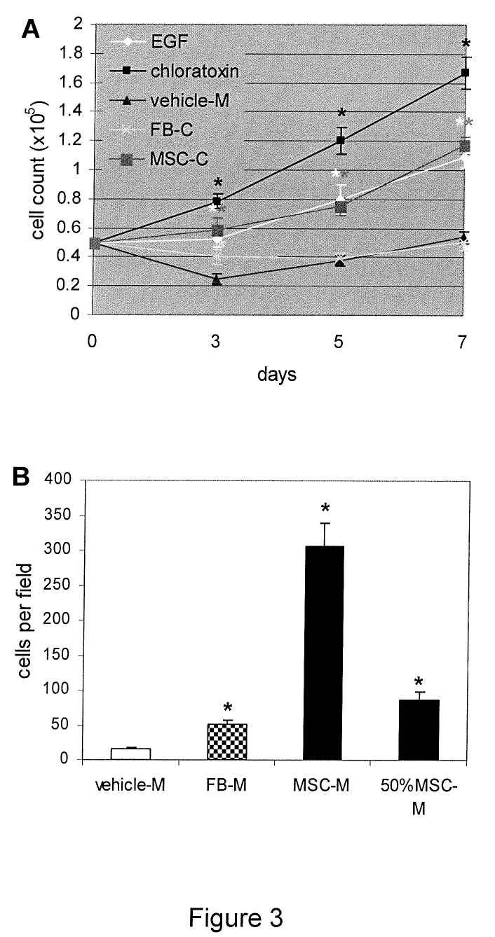

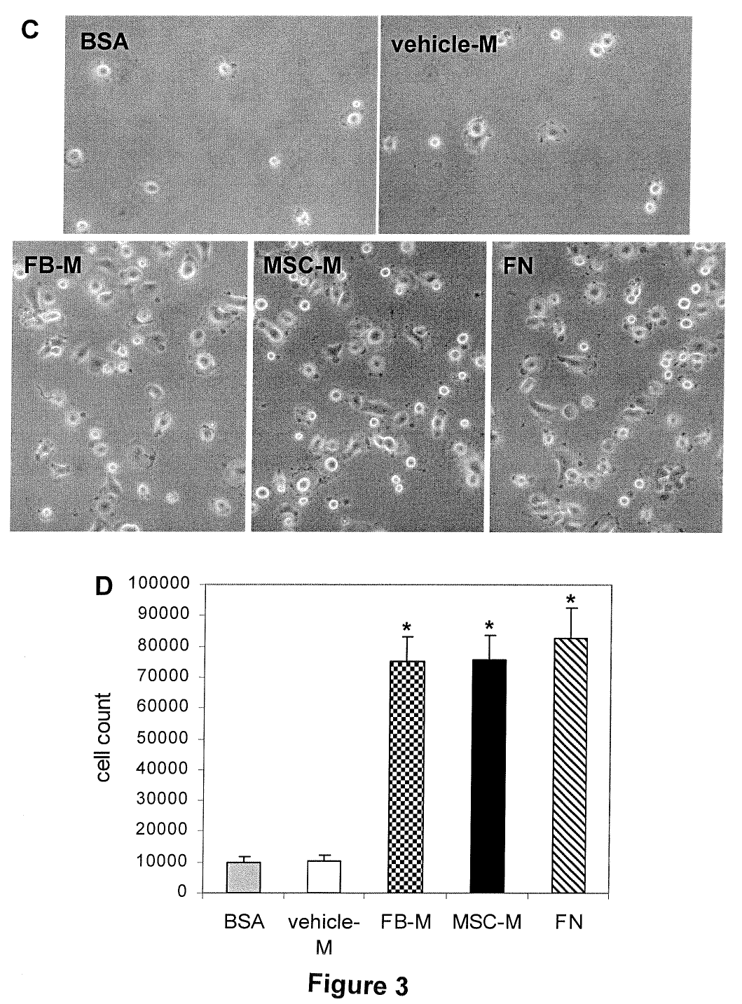

To investigate the indirect influence of BM-MSCs on other cells in the skin, the effects of BM-MSC-conditioned medium were tested on the behavior of keratinocytes. It was found that BM-MSC-conditioned medium derived from hypoxic treatment significantly promoted dermal keratinocyte proliferation (FIG. 3A) and migration (FIG. 3B) compared to control medium or fibroblast-conditioned medium. Fibroblast-conditioned medium significantly promoted keratinocyte adhesion as did BM-MSC-conditioned medium (FIGS. 3C&D) but showed only a modest effect on keratinocyte proliferation (FIG. 3A, compared to vehicle medium, P<0.05 at day 3, P>0.05 at day 5 and 7).

### BM-MSCs Enhance Angiogenesis

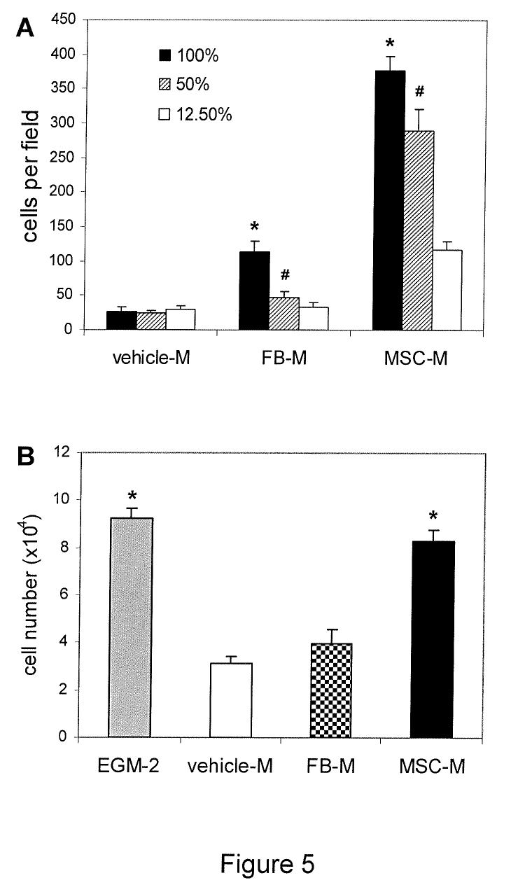

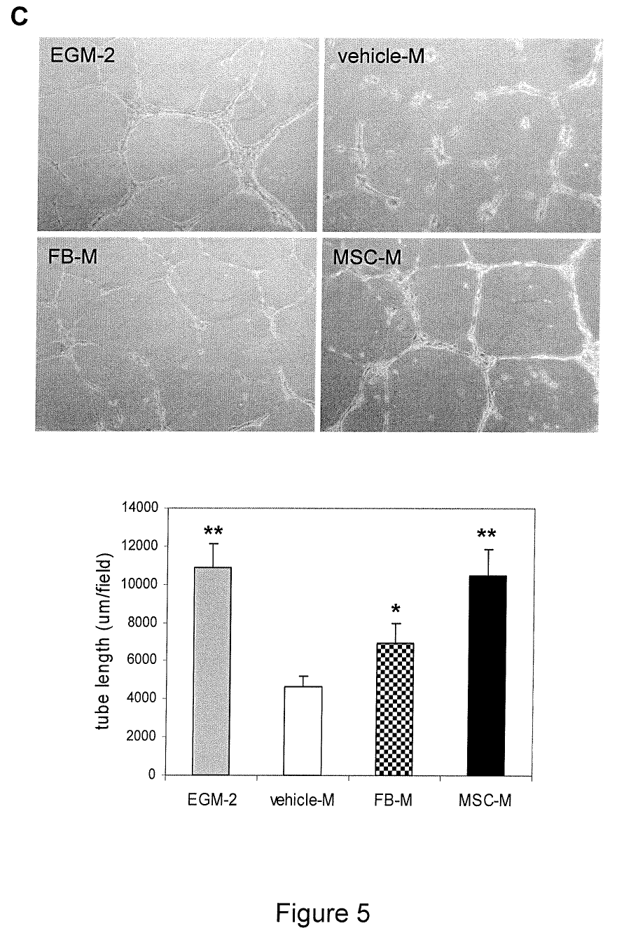

The Balb/C mouse skin is thin and semitransparent, which allows macroscopic visualization of blood vessels in the skin. In the day 7 sham and FB wounds in Balb/C mice, blood vessels were seen clearly in the skin surrounding the wounds, but were limited in the wounds. In contrast, in the wounds of MSC group, vessels and their fine branches extended into the wound forming networks (FIG. 4A). Immunohistological staining of tissue sections for endothelial protein CD31 or vWF showed increased vasculature in BM-MSC-treated wounds at day 7 and 14 compared to vehicle medium- or fibroblast-treated wounds (FIG. 4B). Capillary densities in day 14 wounds were assessed morphometrically after immunohistochemical staining for CD31. As shown in FIG. 4C, capillary density was significantly higher in BM-MSC-treated wounds (771±55/mm²) than in vehicle medium-(357±51/mm²) or fibroblast-(398±44/mm²) treated wounds (n=5, P<0.001). To determine if BM-MSCs enhance angiogenesis through a paracrine effect, HUVECs in BM-MSC-conditioned medium was cultured and it was found that BM-MSC-conditioned medium significantly enhanced HUVEC migration (FIG. 5A), growth (FIG. 5B), and tube formation on Matrigel (FIG. 5C) compared to control medium or fibroblast-conditioned medium.

### Paracrine Effect of BM-MSCs in Wound Healing

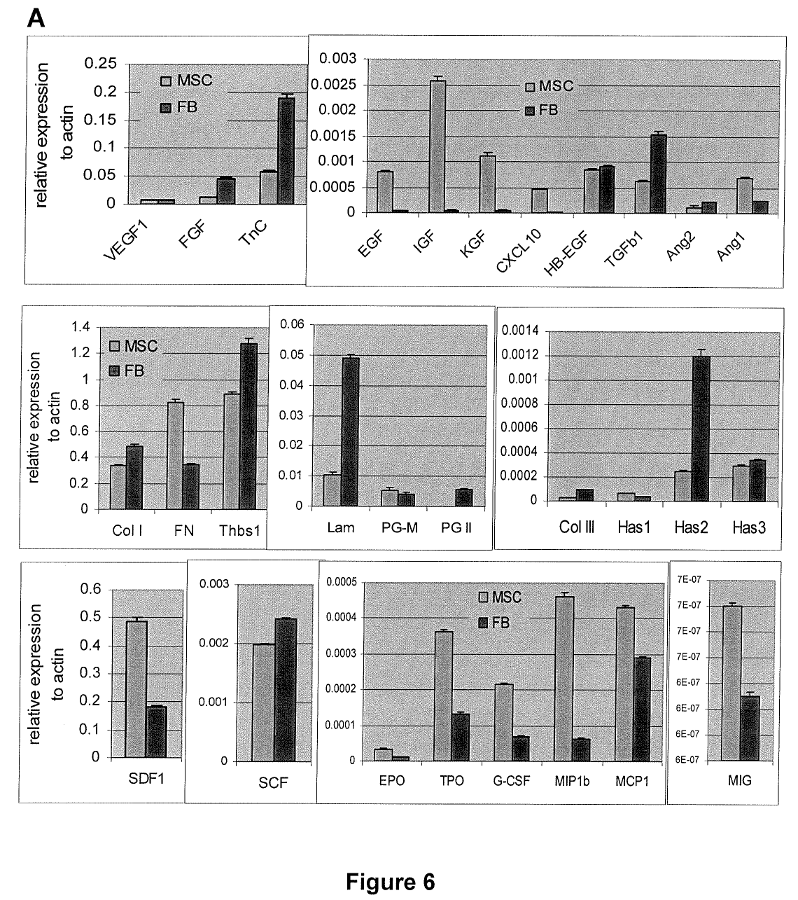

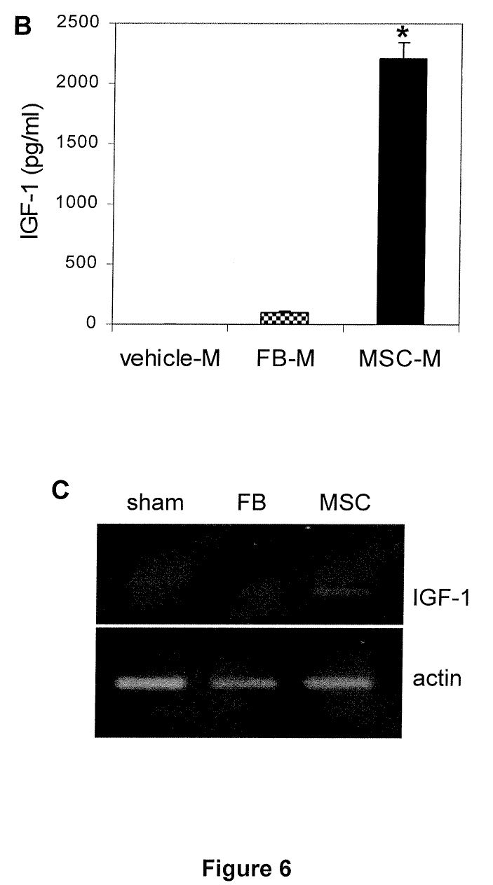

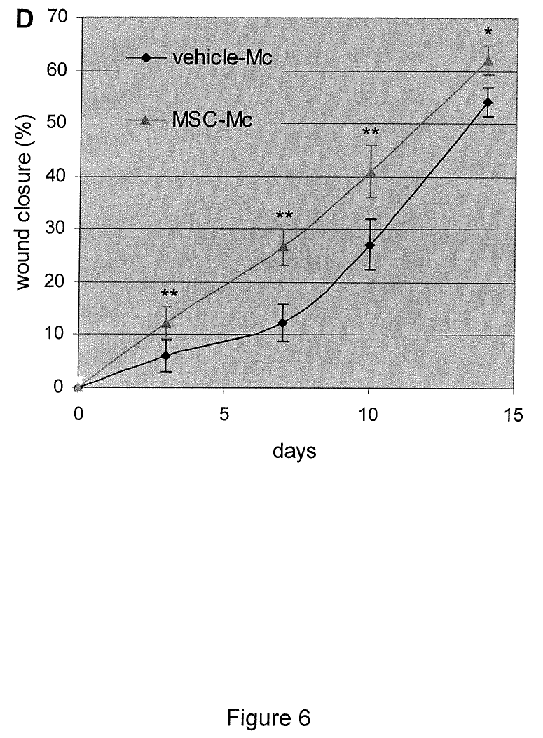

The in vitro tests suggest that BM-MSCs release factors that affect growth, adhesion, migration and angiogenesis of keratinocytes and endothelial cells. To determine the mediators potentially involved, the mRNA expression levels of growth factors, chemokines and adhesion molecules in BM-MSCs after hypoxic treatment compared to neonatal dermal fibroblasts by Real-Time PCR was examined. The analysis revealed that BM-MSCs differentially expressed significantly greater amounts of growth factors such as epidermal growth factor (EGF, 15-fold), keratinocyte growth factor (KGF, 21-fold) and insulin-like growth factor-1 (IGF-1.49-fold) (FIG. 6A) but lower amounts of transforming growth factor (TGF)-β1 (−2.5 fold). While both BM-MSCs and fibroblasts expressed high levels of vascular endothelial growth factor (VEGF)-1, angiopoietin (Ang) 1/Ang2 ratio was a greater in BM-MSCs (5.7) than in fibroblasts (1.07). Among several ECM molecules examined, fibronectin expression in BM-MSCs was higher (2.4-fold). Moreover, BM-MSCs expressed significantly higher amounts of chemoattractants such as stromal derived factor (SDF)-1 (2.7-fold), macrophage inflammatory protein (MIP)-1b (7.3-fold) and monokine induced by gamma interferon (MIG) (2.8-fold) than fibroblasts (FIG. 6A). ELISA measurements (IGF-1 Immunoassay kit, Quantikine, R&D Systems) showed high amounts of IGF-1 in BM-MSC-conditioned medium after hypoxic treatment, which was 22 times higher than that in neonatal dermal fibroblast-conditioned medium (FIG. 6B, P<0.0001). RT-PCR analysis of total RNA extracted from the day 7 wounds for IGF1 indicated a dramatically increased expression in BM-MSC-treated wounds than in fibroblast- or vehicle medium-treated wounds (FIG. 6C).

Finally, to examine whether BM-MSC-released factors could contribute to enhanced wound healing, concentrated BM-MSC-conditioned medium (60 μl/wound concentrated from 5 ml medium from a culture of 1 million BM-MSCs) was injected around excisional wounds in Balb/C mice and found that the treatment resulted in significantly accelerated wound closure compared to injection of vehicle control medium (FIG. 6D).

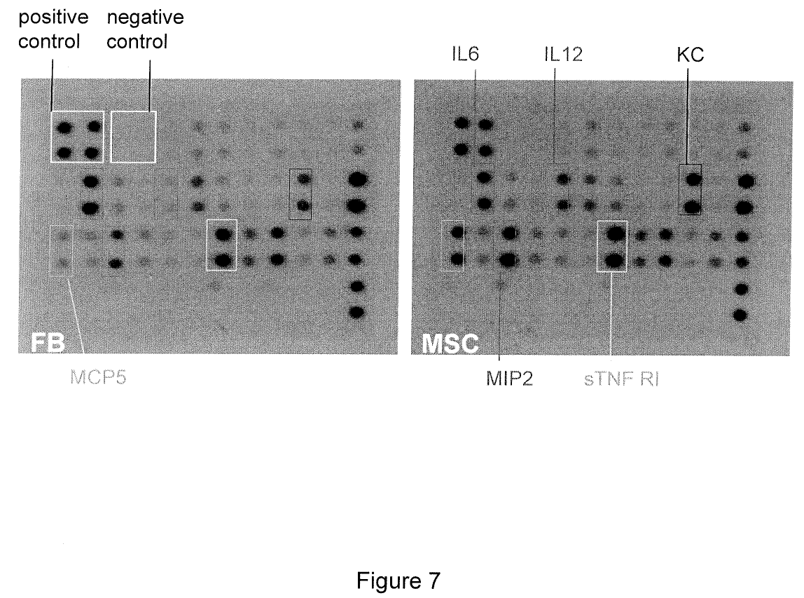

Consistent with our RT-PCR data, antibody array analysis indicated that BM-MSCs expressed differential amounts of several chemokines compared to fibroblasts such as greater amounts of MIP2, IL12, MCP5 and sTNF R1 and less amount of IL6 (FIG. 7). IL6 and tumor necrosis factor (TNF) are potent pro-inflammatory cytokines. Soluble TNF receptor type 1 (sTNF R1) negatively regulates the biological effects of TNF. The discrepancies in chemokine expression between BM-MSCs and fibroblasts may in part explain the differences in cellular components between BM-MSC-treated wounds and fibroblast-treated wounds.

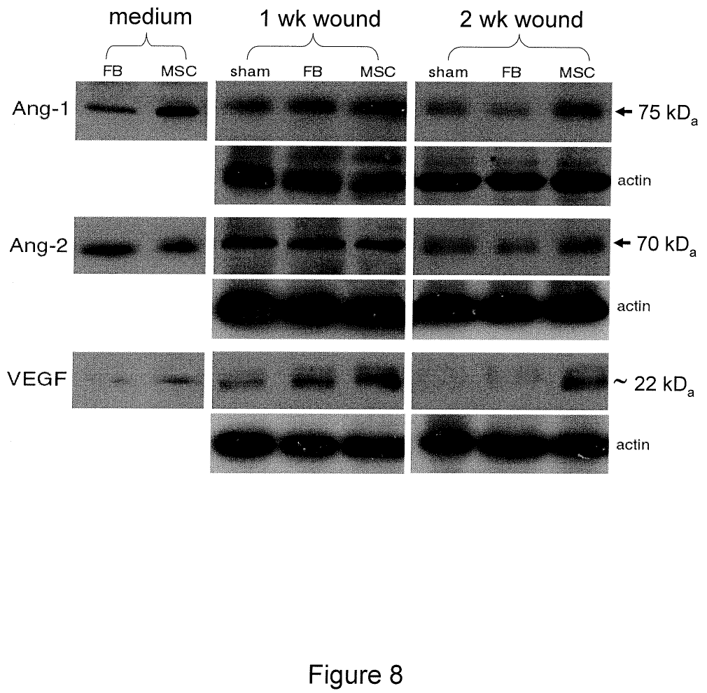

To examine the protein expression levels of angiogenic factors, Western blot analysis of concentrated BM-MSC- or fibroblast-conditioned medium under hypoxic conditions and lysate derived from vehicle medium (sham)-, fibroblast- or BM-MSC-treated wounds was performed. The data showed a greater amount of Ang-1 protein in BM-MSC-conditioned medium and higher levels of Ang-1 in BM-MSC-treated wounds at 7 and 14 days but unchanged amounts of Ang-2 (FIG. 8). Under reducing conditions, the anti-VEGF-a antibody detected a major band of about 22 kDₐ, which corresponds to the molecular size of VEGF164. Of note, much greater amounts of VEGF were detected in BM-MSC-treated wounds compared to vehicle medium- or fibroblast-treated wounds at 7 and 14 days (FIG. 8).

Wounds Treated with BM-MSCs had Increased Levels of Endothelial Progenitor Cells and Macrophages

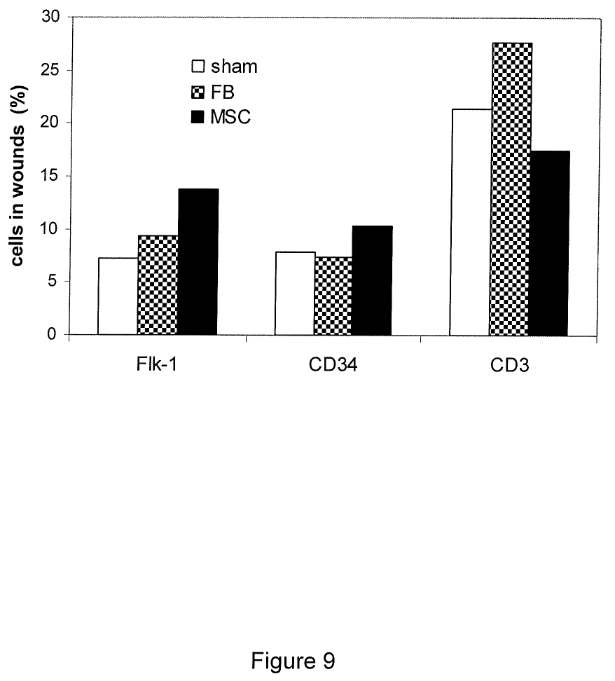

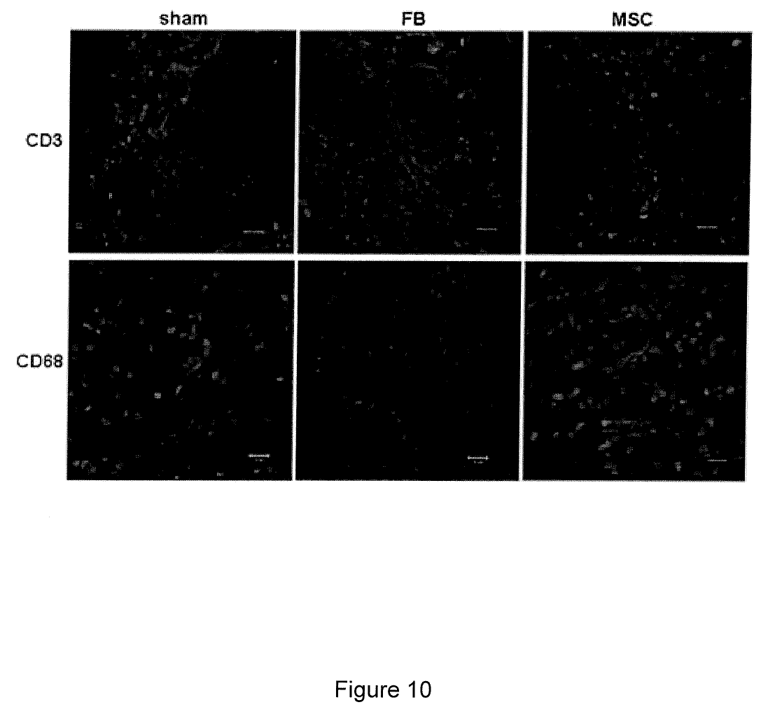

FASC analysis of total cells derived from each wound indicated that BM-MSC-treated wounds had increased numbers of Flk-1 positive and CD34 positive cells but decreased CD3 positive cells compared to vehicle medium (sham)- or fibroblast-treated wounds (FIG. 9). FLK1 and CD34 are characteristic markers for endothelial progenitor cells. Immunoflorescence staining of wound sections demonstrated that BM-MSC-treated wounds had increased CD68 positive macrophages but decreased (CD3) lymphocytes (FIG. 10). These findings suggest that BM-MSCs in the wounds recruit endothelial progenitor cells and macrophages to the wounds while fibroblasts cause increased inflammation, which normally leads to increased scar formation.

The RT-PCR analysis showed that MSC-medium had higher amounts of factors for recruitment of circulating stem cells, such as SDF-1, EPO, TPO and G-CSF. The data show that MSC-treated wounds have higher amounts of endothelial progenitor cells, cells for angiogenesis. In addition, the data also indicated higher amounts of chemoattractive factors for macrophages in MSC-conditioned medium such as MIP and MIG. Both endothelial progenitor cells and macrophages play crucially roles in wound healing. Reduced presence of endothelial progenitor cells are associated with impaired wound healing. In the absence of macrophages, wounds do not close.

### Analysis of MSCs on Keratinocyte Engraftment

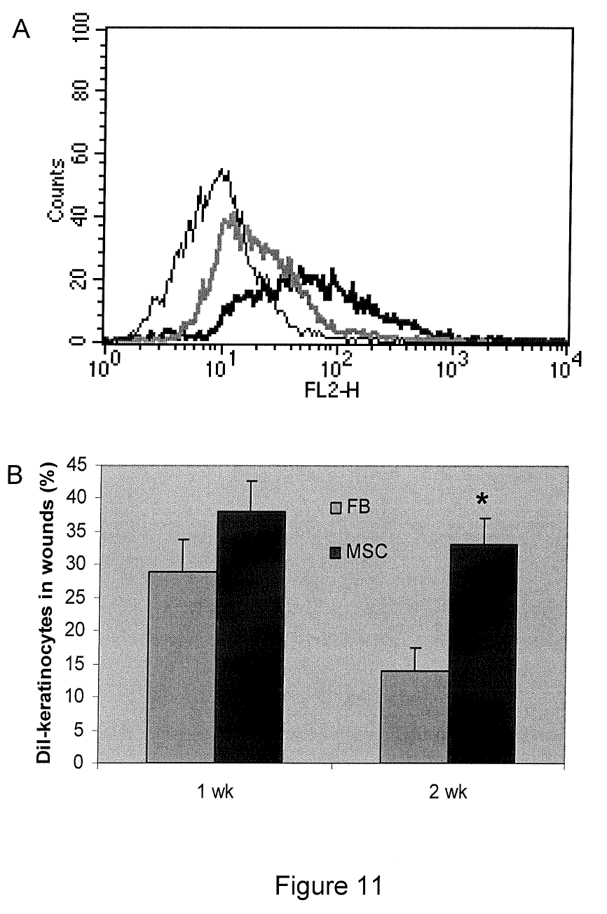

A 10 mm full-thickness skin wound was generated with a punch biopsy on the back in Balb/C mice and the lower chamber of a silicon grafting dome was inserted and secured with suture. One million syngenic BM-MSCs or dermal fibroblasts were mixed with dermal keratinocytes, which were pre-labeled with a fluorescence dye DiI (1:1) in 200 μl Growth Factor Reduced Matrigel (BD), was carefully applied to the wound bed inside the lower chamber. The upper chamber was placed on the lower chamber and fixed with bandage. The dome was removed after one week. Animals were sacrificed at one and two weeks, and the wound along with a small fraction of the surrounding skin was harvested and digested with dispase and hyauluronidase to obtain a single cell suspension. The cells were analyzed on FACS to determine the fractions of DiI-keratinocytes. FACS analysis (FIG. 11) showed that greater amounts of DiI-keratinocytes in wounds received application of a mixture of keratinocytes and BM-MSCs than those received a mixture of keratinocytes in combination with dermal fibroblasts (FIG. 1, n=4, P<0.01). The data suggest that BM-MSCs have a beneficial effect in mediating keratinocyte survival and engraftment.

TABLE 1
Mice at starting of the experiments

| | body weight | glucose | triglyceride | cholesterol |
|---|---|---|---|---|
| | gram | mmol/l | mmol/l | mmol/l |
| db⁺/m⁺ | 22.4 ± 1.6 | 12.6 ± 10.8 | 0.82 ± 0.21 | 1.54 ± 0.17 |
| db⁺/db⁺ | 46.3 ± 3.3** | 44 ± 7.7** | 1.43 ± 0.6* | 2.4 ± 0.59** |

*P < 0.01,
**P < 0.0001

TABLE 2
Criteria for histological scores

| | Epidermal and dermal | | | Angiogenesis |
|---|---|---|---|---|
| score | regeneration | Cell infiltration | Granulation tissue | (day 14 wounds only) |
| 1-3 | Minimal to moderate re- | Wound covered | Granulation | Capillary |
| | epithelialization with or | with thin to | around wound | density <400/mm² |
| | without minimal | moderate cell | edges only | |
| | developing glandular | layer | | |
| | structure formation in | | | |
| | the wound | | | |
| 4-7 | Complete re- | Wound covered | Granulation | Capillary |
| | epithelialization with | with thick cell | around wound | density 400-600/mm² |
| | minimal developing | layer | edge and in 30- | |
| | glandular structure | | 50% of wound bed | |
| | formation in the wound | | | |
| 8-10 | Complete re- | Wound covered | Thick granulation | Capillary |
| | epithelialization with | with very thick | around wound | density >600/mm² |
| | considerable developing | and densely | edge and in >50% | |
| | glandular structure | populated cell | of wound bed | |
| | formation in the wound | layer | | |

TABLE 3
Murine primers for Real-Time PCR

| | |
|---|---|
| FORWARD | REVERSE |

| | | | |
|---|---|---|---|
| Vascular endothelial grwoth factor-a | VEGFa | AGAGCAACATCACCATGCAG | CAGTGAACGCTCCAGGATTT |
| Epidermal growth factor | EGF | AGCTGTGTCTTCTTCACT | TGgGGTCACCTGCTTTAAC |
| keratinocyte growth factor | KGF | CTTCCAATGAGGTCAGCAA | CCAtaAAtCAACAGGCAAAA |
| Isulin-like growth factor | IGF | GGTGGtTTATGAATGGTT | AGGGtGTGtCTAATGGAG |
| heparin-binding EGF-like growth factor | HB-EGF | AAAAGAAGAAGAAAGGAAAGGG | TGCAAGAGGGAGTACGGAA |
| Basic fibroblast growth factor | bFGF | ATGATGACGACGACGATGA | CTACGGTTTGGTTTGGTGTTG |
| Tranforming growth factor betal | TGFβ1 | TGtTAAAACTGGCATCTGA | GTCtCttAGGAAGTAGGT |
| stromal derived factor | SDF-1 | GTCCTCTTGCTGTCCAGCTC | AGATGCTTGACGTTGGCTCT |
| stem cell factor | SCF | TAATGTTCCCCGCTCTCT | TTTTGCTGtTTTTCttTGCTTT |
| erythropoietin | EPO | ACAGTCCCAGATACCAAA | GGCCTTGCCAAACTTCTATG |
| granulocyte colony stimulating factor | G-CSF | ATCATTCTCTCCACTTCC | GTATTTACCCATCTCCTTCCCT |
| Thrombopoietin-1 | TPO | ACCCCAGACTCCTAAATAAAC | CAGCAGAACAGGGATAGACAAA |
| Monocyte chemotactic protein-1 | MCP-1 | CCCGTAAATCTGAAGCTAA | CACACTGGTCACTCCTACAGAA |
| macrophage inflammatory protein1a | MIP1a | CCAGTCCCTTTTCTGTTC | CtTGGTTGCAGAGTGTCAT |
| macrophage inflammatory protein1b | MIP1b | ACGGgGGTCAATTCTAAG | GCCATTCCTGACTCCACA |
| monokine induced by gama interferon | MIG | ACCAAAAGAAAAAGCAAAAGAG | CCTTGAACGACGACGACT |
| procollagen, type I, alpha 1 | Col1a1 | ATTCGGACTAGACATTGG | GGGTTGTTCGTCTGTTTC |
| procollagen, type III, alpha 1 | Col3a1 | TttAGACAtGAtGAGCTT | ATCTACGTTGGACTGCTGTG |
| Fibronectin-1 | Fn1 | AATCCAGTCCACAGCCATTC | TAGTGGCCACCATGAGTCCT |
| Laminin 1 | Lam1 | AGTGGAAGGAATGGTTCACG | TGCCAGTAGCCAGGAAGACT |
| tenascin c | TnC | CAAGGGAGACAAGGAGAG | TCGTCCAGAAAAACGTCAGA |
| thrombospondin | Thbs1 | ACAAGTCACCCAGTCCTA | GAGTTCACAACCttTACAGA |
| versican | PG-M | TGTGCTTCACTCATCATTTC | GCAGTCCCATAATCCAAACC |
| aggrecan 1 | Agc1 | GAGTGAGAACCTACGGAA | CTGgGGATGTCGCATAAAAGA |
| decorin | PG II | CTGGCACAGCATAAGTATATC | AGCCGAGTAGGAAGCCTTT |
| hyaluronan synthase 1 | Has1 | GGGAGgGGTAATTTATTGA | TAGCAACAGGGAGAAAATGGAG |
| hyaluronan synthase 2 | Has2 | CAAAAAGAGCACCAAGGTT | GTGCAGCTTTCCCTTAGACA |
| hyaluronan synthase 3 | Has3 | GTTTCTTCCCATTCTTCCTC | CCtTGATAATGCCCACCA |
| angiopoietin-2 (Ang-2) | Ang2 | GACTTCCAGAGGACGTGGAAAG | CTCATTGCCCAGCCAGTACTC |
| angiopoietin 1 (Angpt1) | Ang-1 | TTGTGATTCTGGTGATTGTGG | CttGTTTCGCttTATTTTTGT |
| gama interferon-inducible protein-10 | CXCL10 | TGTCCTAGCTCTGTACTGT | AACTTAGAACTGACGAGCCT |
| Actin beta | | ACGGCCAGGTCATCACTATTG | CAAGAAGGAAGGCTGGAAAAGA |

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A method for preventing or treating a skin defect on a subject, comprising administering to the skin defect a composition comprising mesenchymal stem cells, a conditioned medium derived from mesenchymal stem cells, or a combination thereof.

2. The method of claim 1, wherein the skin defect comprises a wrinkle, a scar, an aging spot, or a frown line.

3. The method of claim 1, wherein the skin defect comprises an incision or an ulcer.

4. The method of claim 1, wherein the composition is topically applied to the skin defect.

5. The method of claim 1, wherein the composition is injected into the skin defect.

6. The method of claim 1, wherein the mesenchymal stem cells are derived from bone marrow.

7. The method of claim 1, wherein after the administration of the composition to the skin defect the amount of endothelial progenitor cells, macrophages, or combination thereof increases compared to the amount in the skin defect prior to administration of the composition.

8. The method of claim 1, wherein the conditioned cell medium is derived from mesenchymal stem cells under hypoxic or normoxic conditions.

9. The method of claim 1, wherein the administration of the composition induces or enhances angiogenesis in the skin defect.

10. The method of claim 1, wherein the composition further comprises keratinocytes, fibroblasts, endothelial cells, or any combination thereof.

11. A method for inducing or promoting the growth of keratinocytes comprising contacting injured keratinocytes present in the skin defect with a composition comprising mesenchymal stem cells, a conditioned medium derived from mesenchymal stem cells, or a combination thereof.

12. The method of claim 11, wherein the composition further comprises keratinocytes, fibroblasts, endothelial cells, or any combination thereof.

13. A conditioned cell medium produced by the process comprising culturing mesenchymal stem cells under hypoxic or normoxic conditions.

14. A pharmaceutical composition comprising the conditioned cell medium of claim 13, mesenchymal stem cells, or a combination thereof, and a pharmaceutically acceptable carrier.

15. The pharmaceutical composition of claim 14, wherein the composition further comprises keratinocytes, fibroblasts, endothelial cells, or any combination thereof.
